(19) United States
(12) Patent Application Publication
(10) Pub. No.: US 2014/0295539 A1
(43) Pub. Date: Oct. 2, 2014

(54) CONTAINER FOR CULTURING CELLS HAVING NANOSTRUCTURES, AND PREPARATION METHOD THEREOF

(71) Applicants: Dong Sung Kim, Pohang-si (KR); Soo Hong Lee, Seongnam-si (KR); Kyoung Je Cha, Pohang-si (KR); Kwang Sook Park, Seongnam-si (KR)

(72) Inventors: Dong Sung Kim, Pohang-si (KR); Soo Hong Lee, Seongnam-si (KR); Kyoung Je Cha, Pohang-si (KR); Kwang Sook Park, Seongnam-si (KR)

(73) Assignee: POSTECH ACADEMY-INDUSTRY FOUNDATION, Pohang-city (KR)

(21) Appl. No.: 14/353,814

(22) PCT Filed: Mar. 28, 2012
(86) PCT No.: PCT/KR2012/002267

§ 371 Date: Apr. 24, 2014

(30) Foreign Application Priority Data
Nov. 24, 2011 (KR) 10-2011-0123730

(51) Int. Cl. C12M 1/12

(52) U.S. Cl. CPC C12M 25/00
USPC 435/289.1; 264/219; 264/483; 427/2.1; 264/496

(57) ABSTRACT

A container for culturing cells having nanostructures according to the present invention comprises a cell culture surface onto which adult stem cells are adhered so as to be proliferated and differentiated, wherein the cell culture surface comprises nanostructures placed thereon at regular intervals, the nanostructures comprise nano-pillars protruded from the cell culture surface, the width of the nano-pillars is 40-500 nm, and the height of the nano-pillars is 10 nm-1 μm.

### TECHNICAL FIELD

The present invention relates to a cell culture container and a method of manufacturing the same, and more particularly, to a cell culture container in which a nano-structure is included in a cell culture surface to improve attachment, proliferation, and differentiation efficiencies, and a method of manufacturing the same.

### BACKGROUND ART

Recently, cell treatment in which a cell (particularly, a stem cell) in a human body is cultured outside the body and then added back into a patient body to treat disease has expanded. Accordingly, interest in culture methods and culture systems capable of improving proliferation and differentiation efficiencies of the cell by an easy low-priced method is growing. The culture system has a relationship with various devices, in which a cell culture container that can contain a cell culture medium and the cell is one of the most important factors.

In general, many animal cells have attachment dependency, and in this case, after the cell is attached to a bottom by using a cell culture container in which a cell attachable protein is uniformly applied on a flat plate made of plastic or glass, the cell is cultured while being subjected to proliferation and differentiation processes. As described above, the artificially manufactured cell culture container has a surface characteristic that is different from that of an extracellular matrix in which the cell is originally settled, and thus proliferation and differentiation efficiencies of the cell may deteriorate. Actually, the cells are artificially proliferated and then used in clinical treatment, but there is a problem in that inducement of differentiation of various kinds of cells including stem cells and the like for treatment of patients is not easily successful.

The above information disclosed in this Background section is only for enhancement of understanding of the background of the invention and therefore it may contain information that does not form the prior art that is already known in this country to a person of ordinary skill in the art.

### DISCLOSURE

### Technical Problem

The present invention has been made in an effort to provide a nano-structure to a cell culture container to simulate an environment in which a cell is originally settled. Through this, the present invention has been made in an effort to increase attachment, proliferation, and differentiation efficiencies of various kinds of cells including an adult stem cell.

Further, the present invention has been made in an effort to manufacture the cell culture container including the nano-structure by a mass-production mode where the cell culture container and the nano-structure can be simultaneously shaped to reduce a cost required in proliferation and differentiation of the cell.

### Technical Solution

An exemplary embodiment of the present invention provides a cell culture container having a nano-structure, which includes a cell culture surface for allowing an adult stem cell to adhere to perform proliferation and differentiation of the stem cell, in which the cell culture surface includes a nano-structure disposed at a predetermined interval on the cell culture surface, the nano-structure includes a nano-pillar protruding from the cell culture surface, a width of the nano-pillar is in a range between 40 nm and 500 nm, and a height of the nano-pillar is in a range between 10 nm and 1 μm.

The nano-pillar may include a semicircular stereobate and a pillar protruding from the stereobate with a predetermined width and having a semicircular upper portion.

Another exemplary embodiment of the present invention provides a cell culture container having a nano-structure, which includes a cell culture surface for allowing an adult stem cell to adhere to perform proliferation and differentiation of the adult stem cell, in which the cell culture surface includes nano-structures disposed at a predetermined interval on the cell culture surface, the nano-structure includes a nano-pore recessed from the cell culture surface, a width of the nano-pore is in a range between 40 nm and 500 nm, and a depth of the nano-pore is in a range between 10 nm and 1 μm.

The cell culture surface may be formed of at least one of a thermoplastic resin, a thermosetting resin, and an elastic polymer.

The cell culture container may have a surface treated by any one of plasma treatment, ozone treatment, or coating with a cell adhesion improvement material.

Yet another exemplary embodiment of the present invention provides a method of manufacturing a cell culture container having a nano-structure, which includes: forming an alumina template including a preparatory pore by using a two-step aluminum anodization process; forming a polymer material layer on the alumina template and pressing an upper portion of the polymer material layer by the alumina template to form a polymer template; forming a seed layer on surfaces of the polymer template and the alumina template; plating a metal on the seed layer and then removing the polymer template and the alumina template to form a metal mold; and forming a cell culture surface of a polymer material, on which a nano-structure is formed, by forming a cell culture polymer material layer on the metal mold and then removing the metal mold.

The metal mold may include at least one of nickel, iron, copper, silver, gold, and a zinc-tin-lead alloy.

The forming of the cell culture surface may further include forming a first mold including a cavity, equipping the metal mold in the cavity, aligning a second mold to be spaced apart from the first mold at a predetermined interval, injecting a resin for forming the cell culture polymer material layer between the first mold and the second mold, and curing the resin and then removing the first mold and the second mold to complete the cell culture container in which the cell culture surface is formed on an inside bottom thereof.

The resin may be formed of at least one of a thermoplastic resin, a thermosetting resin, and an elastic polymer.

The method may further include treating a surface of the cell culture container by any one of plasma treatment, ozone treatment, and coating with a cell adhesion improvement material.

The forming of the culture surface may be performed by any one of injection molding, hot embossing, UV-molding, and casting.

### Advantageous Effects

According to the exemplary embodiments of the present invention, in a cell culture container, it is possible to allow a nano-structure to affect proliferation and differentiation of a cell to induce differentiation of a stem cell into a specific cell or increase efficiency thereof.

Further, it is possible to mass-produce the cell culture container including the nano-structure to reduce cost and time for cell culture.

### DESCRIPTION OF THE DRAWINGS

FIG. 1 is a schematic diagram illustrating a cell culture container according to an exemplary embodiment of the present invention.

FIG. 2 is a view illustrating expansion of a cell culture surface formed on one surface of the cell culture container according to the exemplary embodiment of the present invention.

FIG. 3 is a cross-sectional view taken along line III-III of FIG. 2.

FIG. 4 is a partially expanded cross-sectional view of a cell culture surface of a cell culture container according to another exemplary embodiment of the present invention.

FIGS. 5 to 9 are views sequentially illustrating a process of manufacturing the cell culture container according to the exemplary embodiment of the present invention.

FIG. 10 is a schematic view of a device for describing a process of manufacturing the cell culture container according to another exemplary embodiment of the present invention.

FIG. 11 is a picture obtained by culturing adipose-derived stem cells in an example of the present invention and a comparative example and observing the adipose-derived stem cells at the 6th day through an optical microscope.

FIG. 12 is a graph illustrating an attachment ratio and a proliferation ratio of the adipose-derived stem cells obtained by culturing the adipose-derived stem cells in one example of the present invention and the comparative example.

FIG. 13 is a picture obtained by comparing local adhesion forms of the adipose-derived stem cells in one example of the present invention and the comparative example.

FIGS. 14 and 15 are pictures and a graph obtained by inducing differentiation of the adipose-derived stem cells into adipocytes in one example of the present invention and the comparative example for comparison.

FIGS. 16 and 17 are pictures and graphs obtained by inducing differentiation of the adipose-derived stem cells into osteocytes in one example of the present invention and the comparative example for comparison.

### MODE FOR INVENTION

The present invention will be described more fully hereinafter with reference to the accompanying drawings, in which exemplary embodiments of the invention are shown. As those skilled in the art would realize, the described embodiments may be modified in various different ways, all without departing from the spirit or scope of the present invention.

In the drawings, the thickness of layers, films, panels, regions, etc., are exaggerated for clarity. Like reference numerals designate like elements throughout the specification. It will be understood that when an element such as a layer, film, region, or substrate is referred to as being “on” another element, it can be directly on the other element or intervening elements may also be present. In contrast, when an element is referred to as being “directly on” another element, there are no intervening elements present.

In describing the present invention, parts that are not related to the description will be omitted. Like reference numerals generally designate like elements throughout the specification.

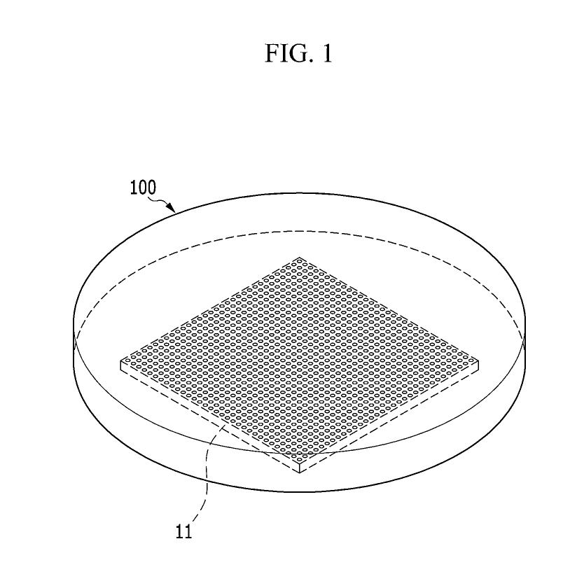

FIG. 1 is a schematic diagram illustrating a cell culture container according to an exemplary embodiment of the present invention.

Referring to FIG. 1, a cell culture container 100 according to the present exemplary embodiment includes a cell culture surface 11. The surface may be an interior bottom surface of the cell culture container 100.

The cell culture surface 11 artificially improves proliferation and differentiation efficiencies of a cell, and induces differentiation in a target direction by allowing a cell to be cultured to adhere to the cell culture surface. Examples of an adult stem cell include a marrow-derived stem cell, a placenta-derived stem cell, an adipose-derived stem cell, and the like, and the cell culture container according to the present exemplary embodiment improves proliferation efficiency of the adult stem cell and improves efficiency of differentiation into a target cell.

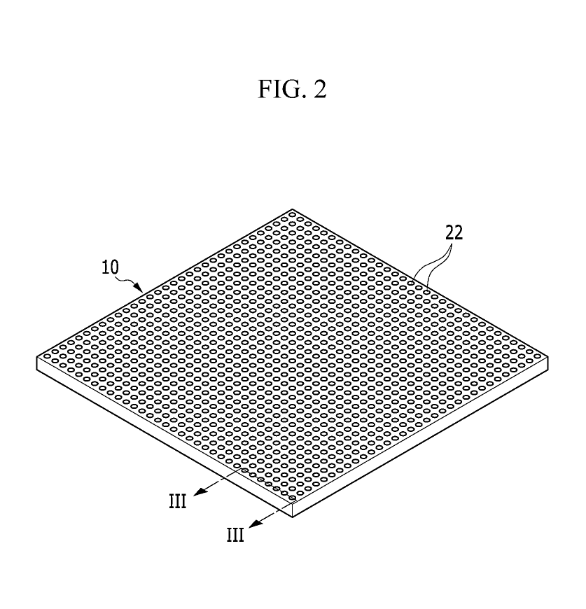

The cell culture surface of the cell culture container according to the exemplary embodiment of the present invention will be described in detail with reference to FIGS. 2 to 4.

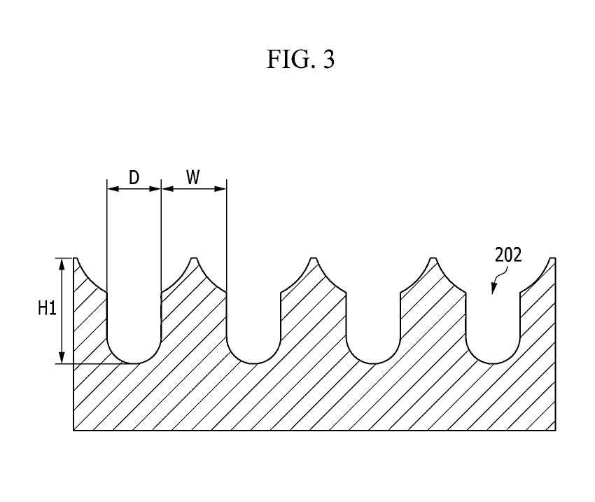

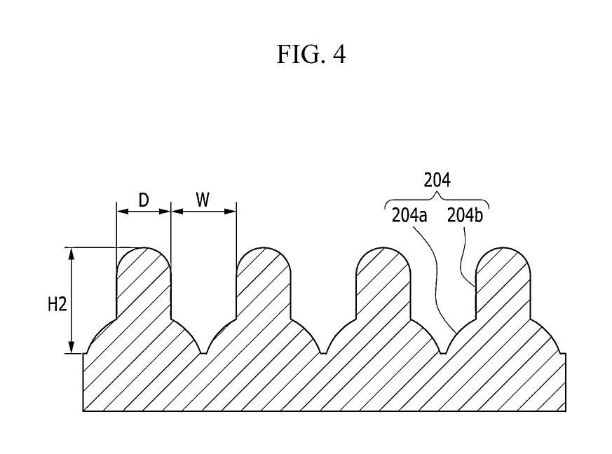

FIG. 2 is a view illustrating expansion of the cell culture surface formed on one surface of the cell culture container according to the exemplary embodiment of the present invention, FIG. 3 is a cross-sectional view taken along line III-III of FIG. 2, and FIG. 4 is a partially expanded cross-sectional view of a cell culture surface of a cell culture container according to another exemplary embodiment of the present invention.

Referring to FIGS. 2 to 4, a nano-structure 22 to which the cell can adhere is formed on the cell culture surface 11.

The nano-structure 22 includes recessed nano-pores, as illustrated in FIG. 3, or nano-pillars protruding from the surface of the cell culture surface 11, as illustrated in FIG. 4.

A nano-pore 202 of FIG. 3 is formed to be recessed to a lower portion of the cell culture surface 11. The nano-pore 202 longitudinally extends in a tube form having a predetermined width, and lower and upper portions of a cross-section thereof have a semicircular shape. The semicircular shape of the upper portion may be formed to have a wider diameter as compared to the semicircular shape of the lower portion, and a cross-section between nano-pores may have a spire structure having a sharp end.

The nano-pores may be formed to have a uniform diameter D in the range of 40 nm to 500 nm, and the diameter may preferably be 200 nm. In addition, a depth H1 may be in the range of 10 nm to 1 μm, and may preferably be 500 nm. In this case, an aspect ratio of the nano-pores may be 1 to 5. Further, in the exemplary embodiment of the present invention, the nano-pores are disposed at a regular interval W of about 500 nm.

In addition, a nano-pillar 204 of FIG. 4 includes a stereobate portion 204a and a protrusion portion 204b protruding from the stereobate portion 204a with a predetermined width, and having a semicircular upper portion.

The nano-pillar 204 of FIG. 4 may be formed to have a uniform diameter D in the range of 40 nm to 500 nm, and the diameter may preferably be 200 nm. In addition, a depth H may be in the range of 10 nm to 1 μm, and may preferably be 500 nm. In this case, an aspect ratio of the nano-pillar may be 1 to 5. Further, in the exemplary embodiment of the present invention, the nano-structures are disposed at a regular interval W of about 500 nm.

The cell culture surface may be formed of polystyrene (PS) that is a thermoplastic resin, and may employ the thermoplastic resin or a thermosetting resin such as polymethyl methacrylate (PMMA) and polycarbonate (PC). Further, the cell culture surface may be formed of an elastic polymer such as polydimethylsiloxane.

In the present invention, a plurality of nano-structures 22 are formed to have a uniform size and are disposed at a regular interval to affect attachment, proliferation, and differentiation of the cell, and thus serves to induce differentiation of the cell in a target direction or increase efficiency thereof. In addition, additional treatment of the surface, such as plasma treatment, ozone treatment, or coating with a cell adhesion improvement material, may be performed over the cell culture surface in order to improve an attachment ability of the cell.

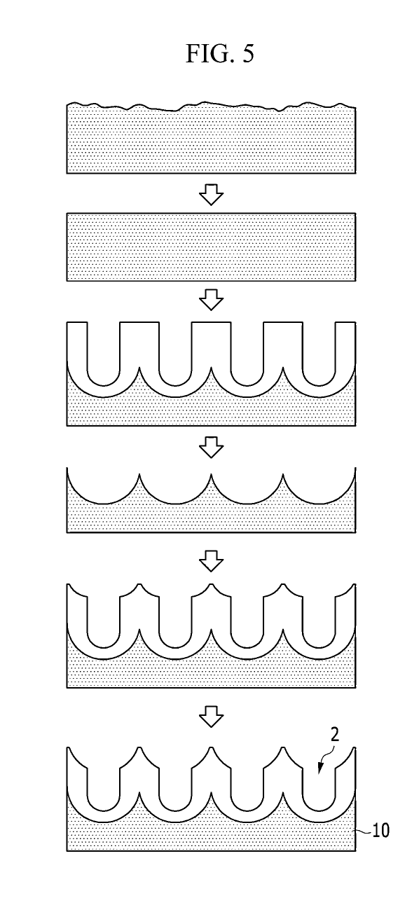

Hereinafter, a method of manufacturing the cell culture container according to the exemplary embodiment of the present invention will be described with reference to FIGS. 5 to 9.

FIGS. 5 to 9 are views sequentially illustrating a process of manufacturing the cell culture container according to the exemplary embodiment of the present invention.

First, as illustrated in FIG. 5, an alumina template 10 is manufactured through a two-step aluminum anodization process. If the aluminum anodization process is performed, anodized alumina is attached onto an aluminum substrate to form an alumina layer including preparatory pores 2.

The preparatory pore 2 may be adjusted by an electrolyte, an anodization voltage, a time, an extension time, and the like used in the aluminum anodization process. In the exemplary embodiment of the present invention, a diameter of the preparatory pore 2 is adjusted to be 200 nm, and a depth is adjusted to be 500 nm by adjusting a two-step aluminum anodization process condition.

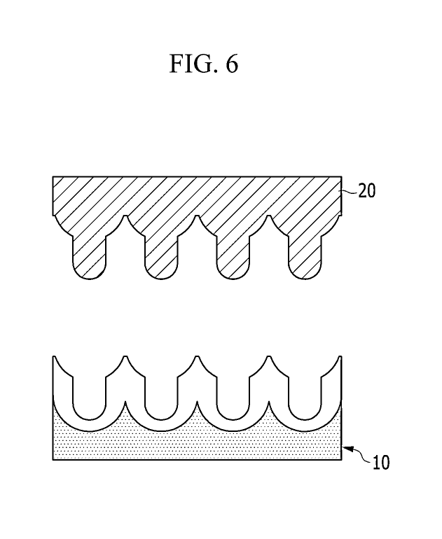

Next, as illustrated in FIG. 6, a shape of the alumina template 10 is transferred on an upper portion of a polymer material layer through a hot embossing process. Thereafter, the alumina template 10 is removed to complete a polymer template 20 formed of a polymer material.

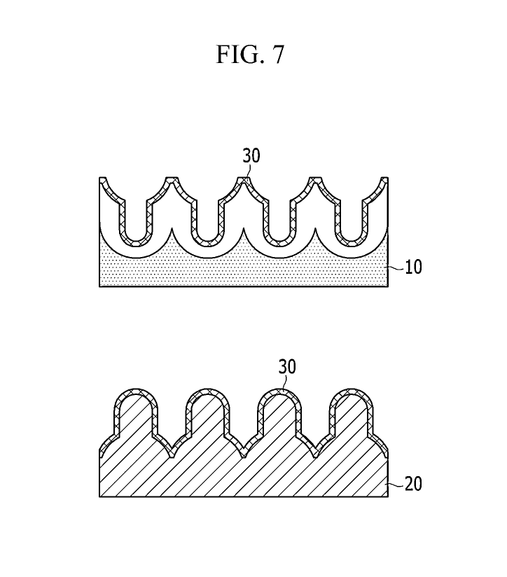

Next, as illustrated in FIG. 7, a seed layer 30 is formed on the alumina template 10 and the polymer template 20. The seed layer 30 may be formed by depositing gold, copper, or nickel that is an electrically conductive material by a method such as CVD (chemical vapor deposition) or ALD (atomic layer deposition). The seed layer is formed in a thickness of about 20 nm.

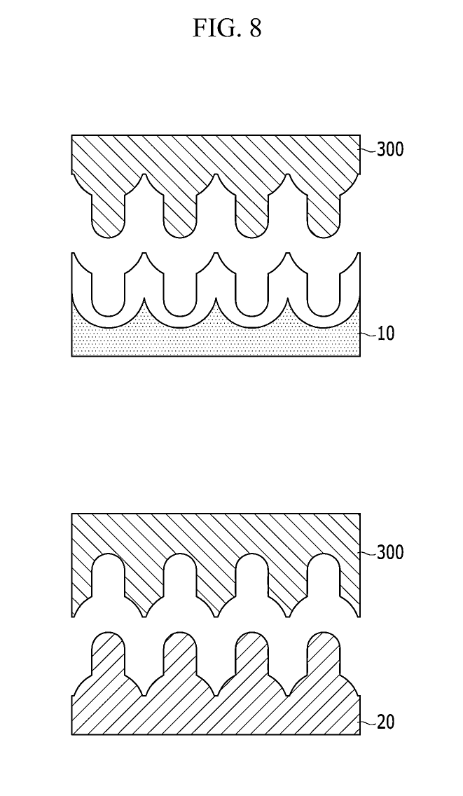

Next, as illustrated in FIG. 8, a metal is plated on the alumina template 10 and the polymer template 20 on which the seed layer 30 is deposited.

The metal is preferably a metal having an excellent abrasion property due to hardness that is higher than that of aluminum, and for example, nickel, iron, copper, silver, gold, a zinc tin-lead alloy, and the like may be used, but the metal is not limited thereto.

In the exemplary embodiment of the present invention, a nickel plating process may be performed, and the nickel plating process is performed by using a nickel plating solution under conditions of a temperature of 50 to 55° C. and pH of 3.7 to 4.2. Further, the nickel plating process is performed at a current density of 1 mA/m² or less, and in this case, in order to minimize residual stress occurring in the course of the plating process, the plating process is performed while the current density is stepwisely increased.

Subsequently, the alumina template 10 and the polymer template 20 are removed from a metal plating layer to obtain a metal mold 300.

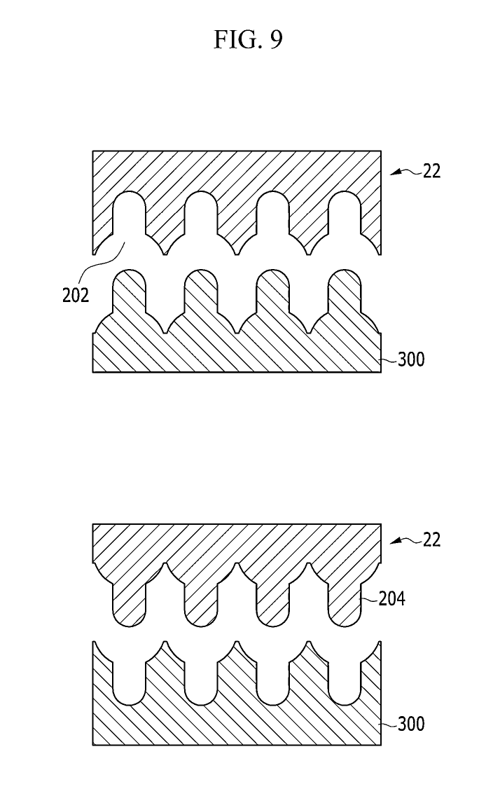

Next, as illustrated in FIG. 9, a substrate including the cell culture surface 11 on which the nano-structure 22 is formed is manufactured by using the metal mold 300.

If a cell culture polymer material layer is formed on the metal mold 300 and then pressed by a hot embossing method, an upper portion of the polymer material layer is transferred the same form as an upper portion of the metal mold 300.

In the exemplary embodiment of the present invention, the polymer material layer may be formed of polystyrene. In this case, pressing is performed at an embossing temperature that is higher than a glass transition temperature (Tg) of the polystyrene by 5 to 20° C. and an embossing pressure in the range of 5 to 10 MPa.

In the exemplary embodiment of the present invention, the cell culture surface employs polystyrene, but is not limited thereto. That is, the cell culture surface may be formed by using the thermoplastic resin or the thermosetting resin in addition to polystyrene, and may be formed by an elastic polymer such as polydimethylsiloxane.

Meanwhile, in the exemplary embodiment of the present invention, a substrate including the cell culture surface formed of a cell culture polymer material is not separately manufactured to be attached to the cell culture container, but may be integrally formed with the cell culture container.

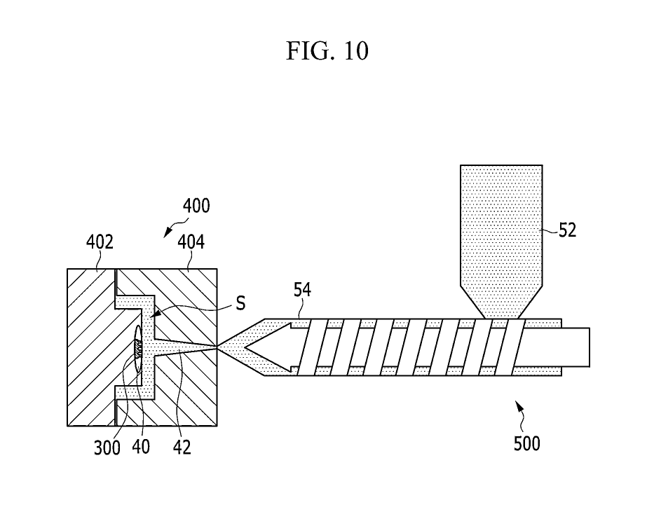

This will be specifically described with reference to FIG. 10.

FIG. 10 is a schematic view of a device for describing a process of manufacturing the cell culture container according to another exemplary embodiment of the present invention.

As illustrated in FIG. 10, a mold 400 including a cavity 40 is prepared.

The mold 400 includes a first mold 402 including the cavity 40, and a second mold 404 disposed to be interlocked with the first mold 402 with a predetermined interval S. In the second mold 404, an inlet 42 through which a resin is injected is formed.

Thereafter, the metal mold 300 formed by the method of FIGS. 5 to 9 is disposed in the cavity 40.

In addition, a shaping resin for forming the cell culture container is injected through the inlet 42 of the second mold 404.

The resin is injected through a resin injection device 500, and the resin injection device 500 includes a hopper 52 in which the resin is contained, and a cylinder 54 connected to a lower portion of the hopper 52 and including a nozzle (not illustrated) that can be inserted into the inlet 42 of the mold. A screw (not illustrated) for moving the resin is positioned in the cylinder.

If the resin is supplied from the hopper 52 to the inside of the cylinder 54, the resin is heated through a heater in the cylinder 54 to be in a fluidized state. Then, the resin moves toward the nozzle by the screw, and the resin in the fluidized state is injected through the nozzle into the interval S and the cavity 40 of the mold.

If injection of the resin is finished, the injected resin is cooled to complete the cell culture container.

Since the resin is injected into the interval S between the first mold 402 and the second mold 404, the cell culture container is formed to have a shape of the interval S. In addition, the resin is injected into the cavity 40, and thus the nano-structure is formed on a bottom surface of the cell culture container to have the same shape as the metal mold positioned in the cavity. Accordingly, the cell culture surface including the nano-structure is integrally formed with the cell culture container.

Further, the cell culture surface may be formed by any one of injection molding, hot embossing, UV-molding, and casting.

If the cell culture container is manufactured like the exemplary embodiment of the present invention, since the cell culture surface on which the nano-structure is formed and the container may be integrally formed, a process of manufacturing the cell culture container becomes simple. Accordingly, time and cost may be reduced. It is preferable to use the thermoplastic resin such as polymethyl methacrylate, polystyrene, and polycarbonate as the resin used in the manufacturing method according the present exemplary embodiment.

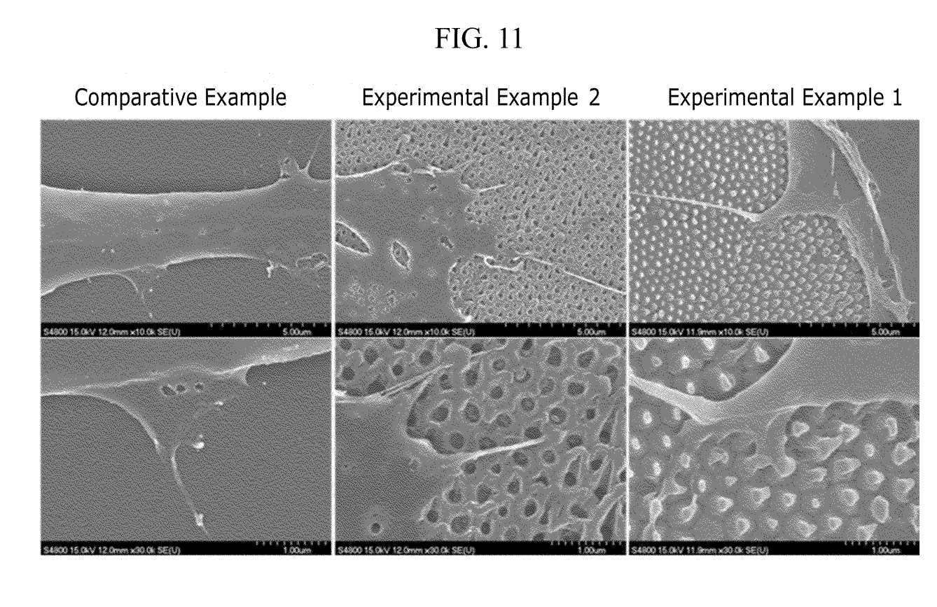

Hereinafter, influence during attachment, proliferation, and differentiation of an adipose-derived stem cell in a cell culture container according to one example of the present invention will be compared to that of a comparative example, and are described with reference to FIGS. 11 to 17.

In Example 1 of the present invention, a cell culture surface of the cell culture container is formed of polystyrene, and the cell culture surface includes a nano-pillar having a diameter of 200 nm and a height of 500 nm as the nano-structure 22.

In addition, in Example 2, a cell culture surface of a cell culture container is formed of polystyrene, and the cell culture surface includes a nano-pore having a diameter of 200 nm and a depth of 500 nm as the nano-structure 22. Each of the nano-structures 22 is formed to be disposed at an interval of 400 nm to 500 nm.

The comparative example is a case where the same experiment is performed in a cell culture container having a flat cell culture surface on which no structure is formed.

First, FIG. 11 shows pictures obtained by culturing adipose-derived stem cells in the present examples and the comparative example and observing the adipose-derived stem cells at the 6th day through an optical microscope.

Referring to FIG. 11, in the comparative example, the cell relatively widely formed podia on a surface. In addition, in the present Examples 1 and 2, it can be confirmed that the adipose-derived stem cell is attached to a protrusion portion of the nano-structure and narrowly formed podia. Accordingly, it can be seen that the adipose-derived stem cell is successfully proliferated in the present Examples 1 and 2.

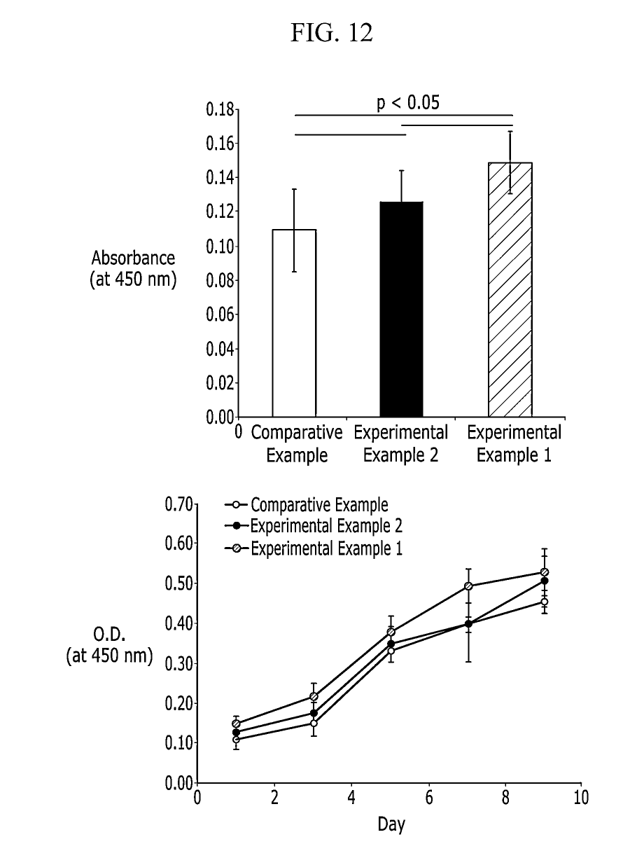

FIG. 12 is a graph illustrating an attachment ratio and a proliferation ratio of the adipose-derived stem cells in the comparative example and the examples of the present invention.

Referring to FIG. 12, it can be seen that the cell attachment ratios of the adipose-derived stem cells aligned in Examples 1 and 2 of the present invention are increased by about 10% and 30% as compared to those of the comparative example. Further, it was observed that in both the present examples, the proliferation ratio was steadily increased as time passed.

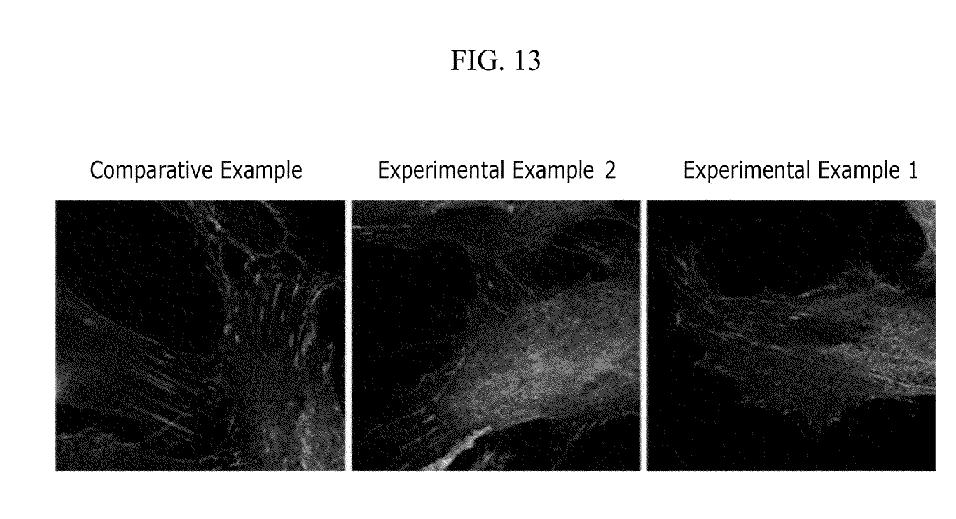

FIG. 13 shows pictures obtained by comparing local adhesion forms of the adipose-derived stem cells in the examples of the present invention and the comparative example.

Referring to FIG. 13, in all of the comparative example and Examples 1 and 2, the adipose-derived stem cell forms a small number of local adhesion sites in the nano-pore structure and forms a small number of cell frames. On the other hand, it can be confirmed that the adipose-derived stem cell forms many small local adhesion sites in the nano-pillar structure and the degree of formation of cell frames is increased.

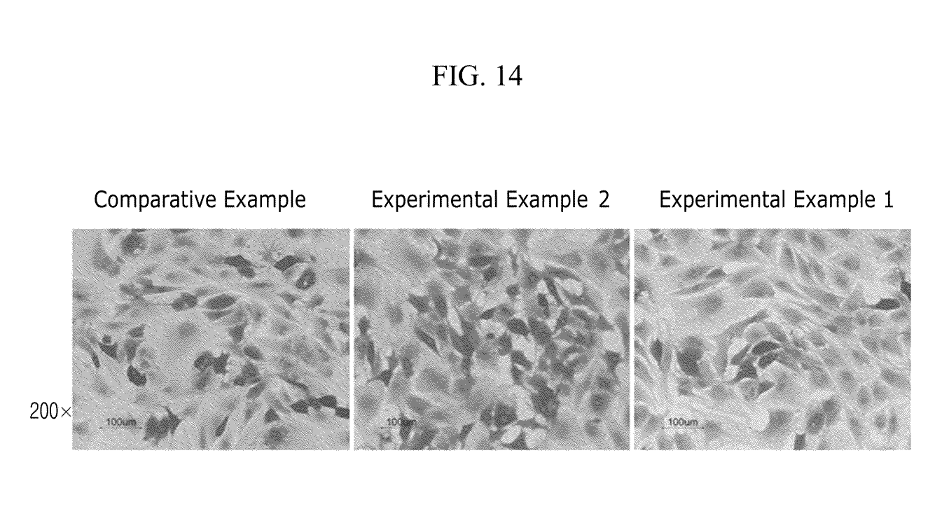

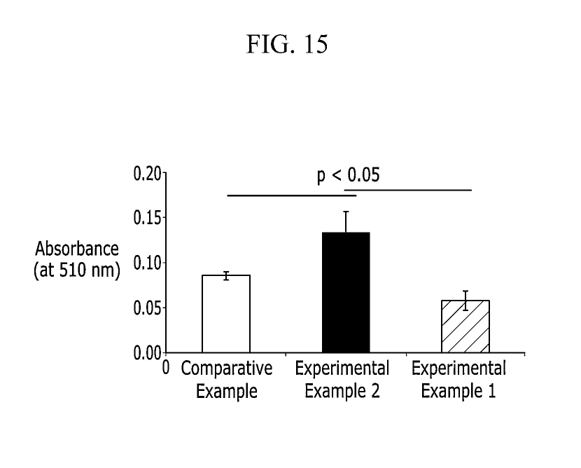

FIGS. 14 and 15 show pictures a graph obtained by inducing differentiation of the adipose-derived stem cells into adipocytes in the examples of the present invention and the comparative example for comparison.

Referring to FIGS. 14 and 15, it can be confirmed that in Example 2 having the nano-pore, differentiation efficiency of the adipose-derived stem cell into the adipocyte is relatively high as compared to that of the adipose-derived stem cells cultured in Example 1 having the nano-pillar and the comparative example.

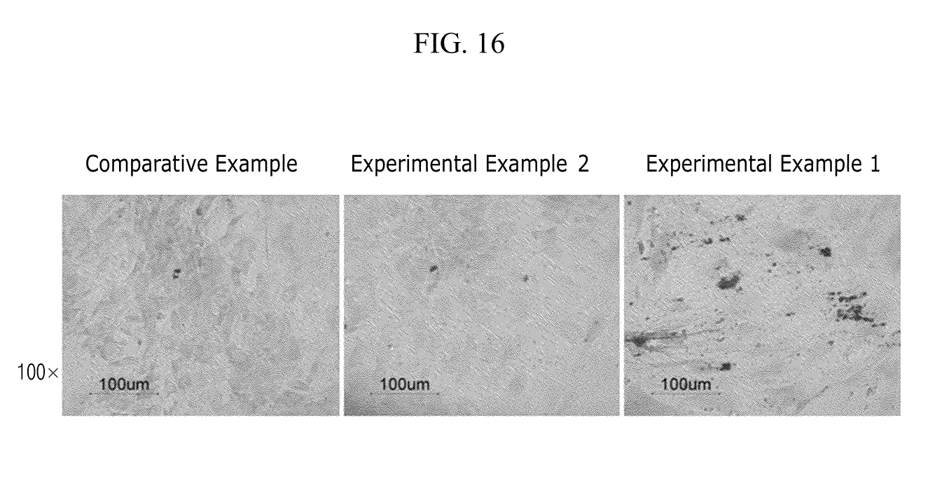

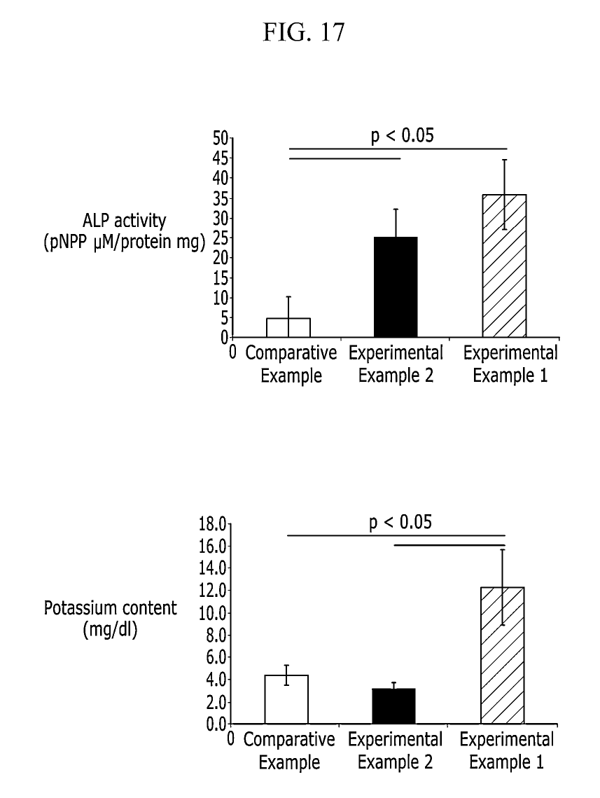

FIGS. 16 and 17 are pictures and graphs obtained by inducing differentiation of the adipose-derived stem cells into osteocytes in the comparative example and Examples 1 and 2 for comparison.

Referring to FIGS. 16 and 17, it can be confirmed that in Example 1 having the nano-pillar, differentiation efficiency of the adipose-derived stem cell into the osteocyte is relatively high as compared to that of the adipose-derived stem cells cultured in Example 2 having the nano-pore and the comparative example.

As seen through FIGS. 11 to 17, when the case where the adult stem cell is cultured on the cell culture surface on which the nano-pore type having the diameter of 200 nm and the depth of 500 nm and the nano-pillar type having the diameter of 200 nm and the height of 500 nm are regularly formed is compared to the case of the flat culture surface, an effect of relatively improving the attachment ratio, the proliferation ratio, and differentiation efficiency can be confirmed.

Accordingly, in the case where the cell is cultured by using the cell culture surface including the nano-structure of the nano-pore or nano-pillar structure having an appropriate size, adhesion, proliferation, and differentiation of the cell may be stably induced. In addition, an effect of stable adhesion of the cell to the cell culture surface in a wider area may be obtained. Accordingly, in the case where the adult stem cell is cultured in the cell culture container including this structure, differentiation efficiency of the cell may be increased and many cells may be obtained.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

1. A cell culture container having a nano-structure, which includes a cell culture surface for allowing an adult stem cell to adhere to perform proliferation and differentiation of the stem cell, wherein:
the cell culture surface includes a nano-structure disposed at a predetermined interval on the cell culture surface;
the nano-structure includes a nano-pillar protruding from the cell culture surface;
a width of the nano-pillar is in a range between 40 nm and 500 nm; and
a height of the nano-pillar is in a range between 10 nm and 1 μm.

2. The cell culture container of claim 1, wherein
the nano-pillar includes a semicircular stereobate and a pillar protruding from the stereobate with a predetermined width and having a semicircular upper portion.

3. A cell culture container having a nano-structure, which includes a cell culture surface for allowing an adult stem cell to adhere to perform proliferation and differentiation of the adult stem cell, wherein
the cell culture surface includes nano-structures disposed at a predetermined interval on the cell culture surface,
the nano-structure includes a nano-pore recessed from the cell culture surface,
a width of the nano-pore is in a range between 40 nm and 500 nm, and
a depth of the nano-pore is in a range between 10 nm and 1 μm.

4. The cell culture container of claim 1, wherein
the cell culture surface is formed of at least one of a thermoplastic resin, a thermosetting resin, and an elastic polymer.

5. The cell culture container of claim 4, wherein
the cell culture container has a surface treated by any one of plasma treatment, ozone treatment, or coating with a cell adhesion improvement material.

6. A method of manufacturing a cell culture container having a nano-structure, comprising:
forming an alumina template including a preparatory pore by using a two-step aluminum anodization process;
forming a polymer material layer on the alumina template and pressing an upper portion of the polymer material layer by the alumina template to form a polymer template;
forming a seed layer on surfaces of the polymer template and the alumina template;
plating a metal on the seed layer and then removing the polymer template and the alumina template to form a metal mold; and
forming a cell culture surface of a polymer material, on which a nano-structure is formed, by forming a cell culture polymer material layer on the metal mold and then removing the metal mold.

7. The method of claim 6, wherein
the metal mold includes at least one of nickel, iron, copper, silver, gold, and a zinc-tin-lead alloy.

8. The method of claim 6, wherein
the forming of the cell culture surface further includes
forming a first mold including a cavity,
equipping the metal mold in the cavity,
aligning a second mold to be spaced apart from the first mold at a predetermined interval,
injecting a resin for forming the cell culture polymer material layer between the first mold and the second mold, and
curing the resin and then removing the first mold and the second mold to complete the cell culture container in which the cell culture surface is formed on an inside bottom thereof.

9. The method of claim 8, wherein
the resin is formed of at least one of a thermoplastic resin, a thermosetting resin, and an elastic polymer.

10. The method of claim 6, further comprising
treating a surface of the cell culture container by any one of plasma treatment, ozone treatment, and coating with a cell adhesion improvement material.

11. The method of claim 6, wherein
the forming of the culture surface is performed by
any one of injection molding, hot embossing, UV-molding, and casting.

12. The cell culture container of claim 3, wherein
the cell culture surface is formed of at least one of a thermoplastic resin, a thermosetting resin, and an elastic polymer.

13. The cell culture container of claim 12, wherein
the cell culture container has a surface treated by any one of plasma treatment, ozone treatment, or coating with a cell adhesion improvement material.
